(19) United States
(12) Patent Application Publication
(10) Pub. No.: US 2009/0131270 A1
(43) Pub. Date: May 21, 2009

(54) METHODS FOR THE DETECTION OF MOLECULAR INTERACTIONS WITHIN CELLS

(75) Inventors: D. Lansing Taylor, Pittsburgh, PA (US); Kenneth A. Giuliano, Pittsburgh, PA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Cellumen, Inc.a Corporation, Pittsburgh, PA (US)

(21) Appl. No.: 11/573,121

(22) PCT Filed: Aug. 2, 2005
(86) PCT No.: PCT/US2005/027919

§ 371 Date: Mar. 11, 2008

Related U.S. Application Data
(60) Provisional application No. 60/598,177, filed on Aug. 2, 2004.

(51) Int. Cl. C40B 30/06; C12Q 1/02; C12Q 1/68; C12Q 1/48; C12Q 1/42; C12Q 1/37; C12M 1/00; C12N 15/11; G06F 17/30

(52) U.S. Cl. 506/10; 435/29; 435/6; 435/15; 435/21; 435/23; 435/287.1; 536/23.1; 707/102; 707/E17.044

(57) ABSTRACT

The invention provides a method of detecting the effect of an agent of interest on the interaction between two or more polypeptides introduced into a cell. The invention also provides a method for quantifying the interaction between at least two molecules of interest, which are introduced into a cell. A method for quantifying the effects of an agent of interest or the interaction between two molecules of interest on cellular constituents or functions in the same cells is also provided. The above inventive methods can be automatically quantified by a device, such as a HCS device, and utilized in the construction of a database.

### CROSS-REFERENCE TO RELATED APPLICATIONS

This patent application claims the benefit of U.S. Provisional Patent Application No. 60/598,177 filed Aug. 2, 2004, the entirety of which is incorporated herein by reference thereto.

### BACKGROUND OF THE INVENTION

Interactions among molecules such as proteins and their role in regulating overall cellular functions are fundamental to biochemistry. Protein-protein interactions, as well as protein interactions with other molecules such as nucleic acids, carbohydrates, and lipids have been recognized as important drug targets (Arkin et al., 2004; Chene, 2004; Toogood, 2002). Such interactions can be correlated, directly or indirectly, to a variety of intracellular events, including signal transduction, metabolism, cell motility, apoptosis, cell cycle regulation, nuclear morphology, cellular DNA content, microtubule-cytoskeleton stability, and histone phosphorylation. For example, the interaction between oncogene MDM2 and the p53 tumor suppressor protein negatively modulates the transcriptional activity and stability of p53. Overexpression of MDM2, found in many human tumors, effectively impairs p53 function. Inhibition of the MDM2-p53 interaction can stabilize p53 and may offer a novel strategy for treating cancer. Through a screening approach, a series of cis-imidazoline analogs have been discovered which activate the p53 pathway in cancer cells, leading to cell cycle arrest, apoptosis, and growth inhibition of tumors (Kussie et al., 1996; Vassilev et al., 2004).

Molecular interactions and the effects of drugs or other treatments on such interactions are currently detected by methods such as in vitro assays where the interactions between purified molecular components are directly measured, two-hybrid systems and variants thereof, in vivo assays where a protein-protein interaction is directly sensed and reported (e.g., fluorescence resonance energy transfer (FRET) between two labeled proteins; incorporation of labeled molecules and detection via antibodies), prediction-based approaches where libraries of 3D protein structures are scanned for potential protein interaction sites based on data sets composed of known protein-protein or protein-ligand interaction structures, and protein tagging and purification of protein-protein complexes followed by mass spectroscopy analysis (Bantscheff et al., 2004; Bauer et al., 2003; Zhu et al., 2003). These methods, however, have numerous disadvantages. For example, low sensitivity of detection, large time requirements for assays, the need to construct multiple chimeric proteins, the inability to monitor molecular binding and its effects in live cells, and the need for specialized and expensive equipment are all limitations on current detection methods. See, for example, U.S. Pat. Nos. 6,902,883, 6,727,071, 6,671,624, 6,620,591, 6,518,021, United States patent application publications 2003/0040012, 2003/0104479, 2003/0143634, 2004//0018504, international patent publication WO 03/012068, and Rubinfeld et al. Thus, improved reagents and methods for detecting and measuring molecular binding events and their effects on other cellular functions are needed.

The development of reagents and assays with the ability to measure molecular binding in a live cell would represent a significant advance in the field. Chimeric proteins having detectable signals could be used to quantify molecular binding events and link those events to other cellular constituents and functions. In addition, high content screening (HCS) technology could be utilized to screen and analyze large numbers of molecular interactions in both live and fixed endpoint assays in cells. HCS assays automate the extraction of multicolor fluorescence information derived from specific fluorescence-based reagents incorporated into cells (Abraham et al., 2004; Giuliano et al., 1997; Giuliano et al., 2003b). HCS technology utilizes an optical system to provide more detailed information about the temporal and spatial dynamics of cell constituents and processes, when compared with standard high throughput screens (Farkas et al., 1993; Giuliano et al., 1997). HCS can be used to conduct a multiplexed assay which can be utilized to both analyze the effects of various agents on molecular interactions and measure the effect on other cellular functions in the same assay. Development of a multiplexed HCS assay which utilizes flexible reagents to detect molecular interactions and their impact on cellular functions would be a powerful tool for drug discovery.

### BRIEF SUMMARY OF THE INVENTION

The invention provides a method of detecting the effect of an agent of interest on the interaction between two or more polypeptides, which are introduced into a cell. The interaction between the polypeptides, endogenous proteins, or combinations thereof, and the disruption of said interactions by an agent of interest can be detected and quantified using a variety of methods involving luminescence or fluorescence. One or more of the polypeptides can be biosensors having an interaction domain, detection domain, localization domain, or combinations thereof. The invention also provides a method for quantifying the interaction between at least two molecules of interest, which are introduced into a cell, where at least one of the molecules has a reporter function which can be used to quantify the interaction. The invention further provides a method for quantifying the effect of an agent of interest or the interaction between two molecules of interest on cellular constituents or functions in the same cells. The above inventive methods can be automatically quantified by a device, such as a HCS device. In addition, any of the above methods can be utilized to create a database of quantified comparisons. These and other advantages of the invention, as well as additional inventive features, will be apparent from the description of the invention provided herein.

### DETAILED DESCRIPTION OF THE INVENTION

This invention provides a method for detecting the effect of an agent of interest on the interaction between two or more polypeptides. The inventive method involves introducing two or more polypeptides into a cell under conditions wherein the polypeptides interact with each other, endogenous proteins, or a combination thereof. The cell is then contacted by the agent of interest, and the interaction between the polypeptides, endogenous proteins, or combination thereof is quantified and compared before and after the addition of the agent of interest.

The polypeptides can comprise, consist of, or consist essentially of proteins, protein fragments, or protein interaction domains. The polypeptides can be prepared by methods known to those of ordinary skill in the art. For example, the polypeptides can be synthesized using solid phase polypeptide synthesis techniques (e.g., Fmoc). Alternatively, the polypeptides can be synthesized using recombinant DNA technology (e.g., using bacterial or eukaryotic expression systems). Accordingly, to facilitate such methods, the invention provides genetic vectors (e.g., plasmids) comprising a sequence encoding the polypeptides, which can be introduced into the cell in accordance with the inventive method. Furthermore, the invention provides the polypeptide in recombinant form.

However it is made, the polypeptide can be isolated and/or purified (or substantially isolated and/or substantially purified). Accordingly, the invention provides the inventive polypeptide in substantially isolated form. The polypeptide can be isolated from other polypeptides as a result of solid phase protein synthesis, for example. Alternatively, the polypeptide can be substantially isolated from other proteins after cell lysis from recombinant production. Standard methods of protein purification (e.g., HPLC) can be employed to substantially purify the inventive polypeptide.

One or more of the polypeptides for use in the inventive method can be a biosensor comprising, consisting of, or consisting essentially of an interaction domain, a localization domain, a reporter domain, or a combination thereof. A biosensor can consist of at least one, more preferably at least three of the above mentioned domains, which are desirably operably linked.

In some preferred embodiments, the biosensor polypeptide can be as described in United States published patent application 2003/0104479 or as described in published PCT application WO 03/012068. The interaction domain can encode a sequence which interacts with one or more polypeptides, endogenous proteins, or a combination thereof.

The localization domain can encode a sequence that directs the polypeptide to a particular location within the cell. Such domain, for example, can cause the polypeptide to be localized within the cell to a particular compartment (e.g., nucleus, nucleolus, Golgi apparatus, endoplasmic reticulum, cell surface, cytoplasm, or other organelle). In one embodiment, the localization domain directs the biosensor to a particular location in the cell, and upon interaction with one or more polypeptides, endogenous proteins, or combinations thereof, the biosensor is directed to a different location in the cell. Upon the disruption of the interaction by an agent of interest, the biosensor either redistributes back to the original location, or to a new location. In another embodiment, the biosensor can have more than one localization domain, where the binding of one of more molecules interferes with the localization ability of at least one of the domains, causing the biosensor to distribute to a different location within the cell. In addition, the term location can refer to either a specific location within a cell (e.g., cytoplasm, nucleus, etc.) or it can refer to an even distribution throughout the cell or several locations within the cell. In another embodiment, the inventive method can include multiple biosensors having different localization domains. For example, the interaction between two biosensors can be used to measure the disruption of a specific protein-protein interaction. In untreated cells, the nuclear-cytoplasmic shuttling of a first biosensor having a nuclear localization signal that interacts with a second biosensor having a nuclear export signal can be tuned by balancing the activities of both localization domains such that the distribution is relatively even throughout the nuclear and cytoplasmic compartments. When an experimental treatment (e.g., an agent of interest) disrupts the interaction between both biosensors, the first biosensor redistributes in the cell according to the activity of its localization domain, which in one embodiment, is a predominately nuclear distribution.

Preferably, the localization domain comprises, consists of, or consists essentially of a nuclear export signal (NES) or a nuclear localization signal (NLS). Preferred examples of the localization domain comprise, consist of, or consist essentially of one of the following sequences:

| | |
|---|---|
| KRTADGSEFESPKKARKVE, | (SEQ ID NO:1) |
| QQMGRGSEFEPAAKRAKLDE, | (SEQ ID NO:2) |
| QQMGRGSEFESPKKARKVE, | (SEQ ID NO:3) |
| NSNELALKLAGLDINKTE, | (SEQ ID NO:4) |
| HAEKVAEKLEALSVKEET, | (SEQ ID NO:5) |
| and | |
| PSTRIQQQLGQLTLENLQ. | (SEQ ID NO:6) |

In another embodiment, the polypeptide includes a reporter domain, which allows detection of the biosensor in the cell via any suitable method. Preferably, the reporter domain is fluorescent or luminescent, such as a type of Green Fluorescent Protein (GFP). Alternatively, the reporter domain can be a small epitope tag, such as a myc tag, to allow for better penetration into multiple cellular compartments and to enable the use of multiple luminescent or fluorescent labeling molecules.

In some preferred embodiments, the inventive method can employ multiple biosensors. A preferred example includes a first biosensor having the sequence or a portion thereof of the p53 tumor suppressor protein, and a second biosensor having the sequence or a portion thereof of HDM2, a protein that interacts with p53, such that the first and second biosensor interact with each other within the cell. In addition, each biosensor can contain one or more localization domains, reporter domains, or combinations thereof. Preferred examples of the p53 biosensor comprise, consist of, or consist essentially of one of the following sequences. In the p53 sequences, the GFP is humanized Ptilosarcus GFP (U.S. Pat. No. 6,780,974), the NES is from MAPKAP2, the NLS is from SV40, and the p53 is amino acids 1-131 from human p53. In the HDM2 sequences, the NES is from Annexin II.

Nucleic Acid Sequence of GFP-NES-NLS-p53

(SEQ ID NO:7)

ATGGTGAACCGGAACGTGCTGAAGAACACCGGCCTGAAGGAGATCATGAG
CGCCAAGGCCAGCGTGGAGGGCATCGTGAACAACCACGTGTTCAGCATGG
AGGGCTTCGGCAAGGGCAACGTGCTGTTCGGCAACCAGCTGATGCAGATC
CGGGTGACCAAGGGCGGCCCTCTGCCCTTCGCCTTCGACATCGTGAGCAT
CGCCTTCCAGTACGGCAACCGGACCTTCACCAAGTATCCCGACGACATCG
CCGACTACTTCGTGCAGAGCTTCCCTGCCGGCTTCTTCTACGAGCGGAAC
CTGCGGTTCGAGGACGGCGCCATCGTGGACATCCGGAGCGACATCAGCCT
GGAGGACGACAAGTTCCACTACAAGGTGGAGTACCGCGGCAACGGCTTCC
CTAGCAACGGCCCTGTGATGCAGAAGGCCATCCTGGGCATGGAGCCCAGC
TTCGAGGTGGTGTACATGAACAGCGGCGTGCTGGTGGGCGAGGTGGACCT
GGTGTACAAGCTGGAGAGCGGCAACTACTACAGCTGCCACATGAAGACCT
TCTACCGGAGCAAGGGCGGCGTGAAGGAGTTCCCTGAGTACCACTTCATC
CACCACCGGCTGGAGAAGACCTACGTGGAGGAGGGCAGCTTCGTGGAGCA
GCACGAGACCGCCATCGCCCAGCTGACCACCATCGGCAAGCCTCTGGGCA
GCCTGCACGAGTGGGTGGAATTCACGCGTGGTACCTCTAGACCTCAGACT
CCACTGCACACCAGCCGTGTCCTGAAGGAGGACAAGGAACGATGGGAGGA
TGTCAAGGAGGAGATGACCAGTGCCTTGGCCACGATGTGTGTTGACTATG
AGCAGATCAAGATAAAGAAGATAGAAGACGCATCCCCAAAGAAGAAGCGA
AAGGTGCTCGAGATGGAGGAGCCGCAGTCAGATCCTAGCGTCGAGCCCCC
TCTGAGTCAGGAAACATTTTCAGACCTATGGAAACTACTTCCTGAAAACA
ACGTTCTGTCCCCCTTGCCGTCCCAAGCAATGGATGATTTGATGCTGTCC
CCGGACGATATTGAACAATGGTTCACTGAAGACCCAGGTCCAGATGAAGC
TCCCAGAATGCCAGAGGCTGCTCCCCGCGTGGCCCCTGCACCAGCAGCTC
CTACACCGGCGGCCCCTGCACCAGCCCCCTCCTGGCCCCTGTCATCTTCT
GTCCCTTCCCAGAAAACCTACCAGGGCAGCTACGGTTTCCGTCTGGGCTT
CTTGCATTCTGGGACAGCCAAGTCTGTGACTTGCACGTACTCCCCTGCCC
TCAACAAGATGTTTTGCCAACTGGCCAAGACCTGCTAA
Amino Acid Sequence of GFP-NES-NLS-p53

(SEQ ID NO:8)

MVNRNVLKNTGLKEIMSAKASVEGIVNNHVFSMEGFGKGNVLFGNQLMQI
RVTKGGPLPFAFDIVSIAFQYGNRTFTKYPDDIADYFVQSFPAGFFYERN
LRFEDGAIVDIRSDISLEDDKFHYKVEYRGNGFPSNGPVMQKAILGMEPS
FEVVYMNSGVLVGEVDLVYKLESGNYYSCHMKTFYRSKGGVKEFPEYHFI
HHRLEKTYVEEGSFVEQHETAIAQLTTIGKPLGSLHEWVEFTRGTSRPQT
PLHTSRVLKEDKERWEDVKEEMTSALATMCVDYEQIKIKKIEDASPKKKR
KVLEMEEPQSDPSVEPPLSQETFSDLWKLLPENNVLSPLPSQAMDDLMLS
PDDIEQWFTEDPGPDEAPRMPEAAPRVAPAPAAPTPAAPAPAPSWPLSSS
VPSQKTYQGSYGFRLGFLHSGTAKSVTCTYSPALNKMFCQLAKTC
Nucleic Acid Sequence of HDM2-NES-myc

(SEQ ID NO:9)

ATGTGCAATACCAACATGTCTGTACCTACTGATGGTGCTGTAACCACCTC
ACAGATTCCAGCTTCGGAACAAGAGACCCTGGTTAGACCAAAGCCATTGC
TTTTGAAGTTATTAAAGTCTGTTGGTGCACAAAAAGACACTTATACTATG
AAAGAGGTTCTTTTTTATCTTGGCCAGTATATTATGACTAAACGATTATA
TGATGAGAAGCAACAACATATTGTATATTGTTCAAATGATCTTCTAGGAG
ATTTGTTTGGCGTGCCAAGCTTCTCTGTGAAAGAGCACAGGAAAATATAT
ACCATGATCTACAGGAACTTGGTAGTAGTCAATCAGCAGGAATCATCGGA
CTCAGGTACATCTGTGAGTGAGAACAGGTGTCACCTTGAAGGTGGGAGTG
ATCAAAAGGACCTTGTACAAGAGCTTCAGGAAGAGAAACCTTCATCTTCA
CATTTGGTTTCTAGACCATCTACCTCATCTAGAAGGAGAGCAATTAGTGA
GACAGAAGAAAATTCAGATGAATTATCTGGTGAACGACAAAGAAAACGCC
ACAAATCTGATAGTATTTCCCTTTCCTTTGATGAAAGCCTGGCTCTGTGT
GTAATAAGGGAGATATGTTGTGAAAGAAGCAGTAGCAGTGAATCTACAGG
GACGCCATCGAATCCGGATCTTGATGCTGGTGTAAGTGAACATTCAGGTG
ATTGGTTGGATCAGGATTCAGTTTCAGATCAGTTTAGTGTAGAATTTGAA
GTTGAATCTCTCGACTCAGAAGATTATAGCCTTAGTGAAGAAGGACAAGA
ACTCTCAGATGAAGATGATGAGGTATATCAAGTTACTGTGTATCAGGCAG
GGGAGAGTGATACAGATTCATTTGAAGAAGATCCTGAAATTTCCTTAGCT
GACTATTGGAAATGCACTTCATGCAATGAAATGAATCCCCCCCTTCCATC
ACATTGCAACAGATGTTGGGCCCTTCGTGAGAATTGGCTTCCTGAAGATA
AAGGGAAAGATAAAGGGGAAATCTCTGAGAAAGCCAAACTGGAAAACTCA
ACACAAGCTGAAGAGGGCTTTGATGTTCCTGATTGTAAAAAAACTATAGT
GAATGATTCCAGAGAGTCATGTGTTGAGGAAAATGATGATAAAATTACAC
AAGCTTCACAATCACAAGAAAGTGAAGACTATTCTCAGCCATCAACTTCT
AGTAGCATTATTTATAGCAGCCAAGAAGATGTGAAAGAGTTTGAAAGGGA
AGAAACCCAAGACAAAGAAGAGAGTGTGGAATCTAGTTTGCCCCTTAATG
CCATTGAACCTTGTGTGATTTGTCAAGGTCGACCTAAAAATGGTTGCATT
GTCCATGGCAAAACAGGACATCTTATGGCCTGCTTTACATGTGCAAAGAA
GCTAAAGAAAAGGAATAAGCCCTGCCCAGTATGTAGACAACCAATTCAAA
TGATTGTGCTAACTTATTTCCCCATGTCTACTGTCCACGAAATCCTGTGC
AAGCTCAGCTTGGAGGGTGTTCATTCTACACCCCCAAGTGCCGAACAAAA
ACTCATCTCAGAAGAGGATCTGAATATGCATACCGGTTAG
Amino Acid Sequence of HDM2-NES-myc

(SEQ ID NO:10)

MCNTNMSVPTDGAVTTSQIPASEQETLVRPKPLLLKLLKSVGAQKDTYTM
KEVLFYLGQYIMTKRLYDEKQQHIVYCSNDLLGDLFGVPSFSVKEHRKIY
TMIYRNLVVVNQQESSDSGTSVSENRCHLEGGSDQKDLVQELQEEKPSSS
HLVSRPSTSSRRRAISETEENSDELSGERQRKRHKSDSISLSFDESLALC
VIREICCERSSSSESTGTPSNPDLDAGVSEHSGDWLDQDSVSDQFSVEF
EVESLDSEDYSLSEEGQELSDEDDEVYQVTVYQAGESDTDSFEEDPEISL
ADYWKCTSCNEMNPPLPSHCNRCWALRENWLPEDKGRDKGEISEKAKLEN
STQAEEGFDVPDCKKTIVNDSRESCVEENDDKITQASQSQESEDYSQPST
SSSIIYSSQEDVKEFEREETQDKEESVESSLPLNAIEPCVICQGRPKNGC
IVHGKTGHLMACFTCAKKLKKRNKPCPVCRQPIQMIVLTLTYFPMSTVHE
ILCKLSLEGVHSTPPSAEQKLISEEDLNMHTG
Nucleic Acid Sequence of HDM2-NES

(SEQ ID NO:11)

ATGTGCAATACCAACATGTCTGTACCTACTGATGGTGCTGTAACCACCTC
ACAGATTCCAGCTTCGGAACAAGAGACCCTGGTTAGACCAAAGCCATTGC
TTTTGAAGTTATTAAAGTCTGTTGGTGCACAAAAAGACACTTATACTATG
AAAGAGGTTCTTTTTTATCTTGGCCAGTATATTATGACTAAACGATTATA
TGATGAGAAGCAACAACATATTGTATATTGTTCAAATGATCTTCTAGGAG
ATTTGTTTGGCGTGCCAAGCTTCTCTGTGAAAGAGCACAGGAAAATATAT
ACCATGATCTACAGGAACTTGGTAGTAGTCAATCAGCAGGAATCATCGGA
CTCAGGTACATCTGTGAGTGAGAACAGGTGTCACCTTGAAGGTGGGAGTG
ATCAAAAGGACCTTGTACAAGAGCTTCAGGAAGAGAAACCTTCATCTTCA
CATTTGGTTTCTAGACCATCTACCTCATCTAGAAGGAGAGCAATTAGTGA
GACAGAAGAAAATTCAGATGAATTATCTGGTGAACGACAAAGAAAACGCC
ACAAATCTGATAGTATTTCCCTTTCCTTTGATGAAAGCCTGGCTCTGTGT
GTAATAAGGGAGATATGTTGTGAAAGAAGCAGTAGCAGTGAATCTACAGG
GACGCCATCGAATCCGGATCTTGATGCTGGTGTAAGTGAACATTCAGGTG
ATTGGTTGGATCAGGATTCAGTTTCAGATCAGTTTAGTGTAGAATTTGAA
GTTGAATCTCTCGACTCAGAAGATTATAGCCTTAGTGAAGAAGGACAAGA
ACTCTCAGATGAAGATGATGAGGTATATCAAGTTACTGTGTATCAGGCAG
GGGAGAGTGATACAGATTCATTTGAAGAAGATCCTGAAATTTCCTTAGCT
GACTATTGGAAATGCACTTCATGCAATGAAATGAATCCCCCCCTTCCATC
ACATTGCAACAGATGTTGGGCCCTTCGTGAGAATTGGCTTCCTGAAGATA
AAGGGAAAGATAAAGGGGAAATCTCTGAGAAAGCCAAACTGGAAAACTCA
ACACAAGCTGAAGAGGGCTTTGATGTTCCTGATTGTAAAAAAACTATAGT
GAATGATTCCAGAGAGTCATGTGTTGAGGAAAATGATGATAAAATTACAC
AAGCTTCACAATCACAAGAAAGTGAAGACTATTCTCAGCCATCAACTTCT
AGTAGCATTATTTATAGCAGCCAAGAAGATGTGAAAGAGTTTGAAAGGGA
AGAAACCCAAGACAAAGAAGAGAGTGTGGAATCTAGTTTGCCCCTTAATG
CCATTGAACCTTGTGTGATTTGTCAAGGTCGACCTAAAAATGGTTGCATT
GTCCATGGCAAAACAGGACATCTTATGGCCTGCTTTACATGTGCAAAGAA
GCTAAAGAAAAGGAATAAGCCCTGCCCAGTATGTAGACAACCAATTCAAA
TGATTGTGCTAACTTATTTCCCCATGTCTACTGTCCACGAAATCCTGTGC
AAGCTCAGCTTGGAGGGTGTTCATTCTACACCCCCAAGTGCCTAG
Amino Acid Sequence of HDM2-NES

(SEQ ID NO:12)

MCNTNMSVPTDGAVTTSQIPASEQETLVRPKPLLLKLLKSVGAQKDTYTM
KEVLFYLGQYIMTKRLYDEKQQHIVYCSNDLLGDLFGVPSFSVKEHRKIY
TMIYENLVVVNQQESSDSGTSVSENRCHLEGGSDQKDLVQELQEEKPSSS
HLVSRPSTSSRRRAISETEENSDELSGERQRKRHKSDSISLSFDESLALC
VIREICCERSSSSESTGTPSNPDLDAGVSEHSGDWLDQDSVSDQFSVEFE
VESLDSEDYSLSEEGQELSDEDDEVYQVTVYQAGESDTDSFEEDPEISLA
DYWKCTSCNEMNPPLPSHCNRCWALRENWLPEDKGKDKGEISEKAKLENS
TQAEEGFDVPDCKKTIVNDSRESCVEENDDKITQASQSQESEDYSQPSTS
SSIIYSSQEDVKEFEREETQDKEESVESSLPLNAIEPCVICQGRPKNGCI
VHGKTGHLMACFTCAKKLKKRNKPCPVCRQPIQMIVLTYFPMSTVHEILC
KLSLEGVHSTPPSA

The inventive method can be used to detect the effect of an agent. The agent can be but is not limited to, for example, a chemical, physical stimulus, environmental stimulus, electrical stimulus, or radiation (e.g., heat, light, or other electromagnetic radiation, or radiation from an unstable isotope).

In other embodiments, this invention provides a method for quantifying the interaction between at least two molecules of interest. The inventive method involves introducing each molecule of interest individually into a cell and quantifying the result, then introducing said molecules of interest into a cell concurrently, quantifying the result, and comparing the results before and after the concurrent addition of the molecules. Preferably, at least one of the molecules of interest is a polypeptide. Even more preferably, one of the molecules is the src-homology domain 2 (SH2), or a portion or mutant thereof. In a preferred embodiment, at least one of the molecules of interest is a biosensor, as described above, which may contain an interaction, localization, or reporter domain, or a combination thereof. For example, the biosensor may contain an interaction domain that interacts with a molecule of interest, endogenous protein, or combinations thereof.

The polypeptides and/or molecules of interest can be exogenous, endogenous, or a combination thereof. Exogenous molecules or polypeptides can be introduced into cells by a variety of transfection and amplification techniques familiar to one of skill in the art. In addition, the polypeptides or molecules can be can be prepared outside, then introduced into the cell, which allows for more flexibility in biosensor design such as the use of bright synthetic site-directed fluorescent dyes that can be used with other modes of fluorescence imaging such as steady-state anisotropy that can be easily incorporated into HCS. Examples of methods of introduction include but are not limited to electroporation, fusion with transport peptides such as tat, and high-speed opto-injection (Cyntellect, San Diego, Calif.). In addition, the polypeptides or molecules can be expressed within in the cell under the transcriptional control of an inducible promoter.

Detection and quantification of the biosensors can be quantified by using fluorescence resonance energy transfer, fluorescence anisotropy, rotational difference, fluorescence lifetime change, fluorescence solvent sensitivity, fluorescence quenching, or any other method known to one of skill in the art. The above detection methods can be employed as described in Gough et al., 1993, Bastiaens et al., 1999, and Giuliano et al. 1995.

In preferred embodiments, the quantification step is automatically achieved using a device. For example, the device can have an array of locations which contain multiple cells, where multiple cells in each of the locations containing cells are scanned to obtain luminescent or fluorescent signals from at least one luminescent or fluorescent reporter polypeptide within the cells and the luminescence or fluorescence intensity of the luminescent or fluorescent signals from at least one luminescent or fluorescent reporter polypeptide within a specific location in the cells is measured. The changes induced by the agent or molecule of interest can be automatically calculated. Two preferred methods include comparing a ratio of luminescent or fluorescent signal intensity from at least one luminescent or fluorescent reporter polypeptide in a specific location in the cells to luminescent or fluorescent signal intensity from at least one luminescent or fluorescent reporter polypeptide in a different specific location in the cells; and comparing a difference between luminescent or fluorescent signal intensity from the at least one luminescent or fluorescent reporter polypeptide in a specific location in the cells and luminescent or fluorescent signal intensity from at least one luminescent or fluorescent reporter polypeptide in a different specific location in the cells wherein the changes induced by an agent or molecule of interest indicate an effect of the agent or molecule of interest on the localization of the polypeptide from a first location in the cells to a second specific location in the cells.

In a preferred embodiment, the device uses HCS technology. For example, the technology as described in U.S. Pat. Nos. 6,902,883; 6,727,071; 6,671,624; and 6,620,591 could be used by one having ordinary skill in the art to detect and quantify the inventive method. Preferred devices are the ArrayScan and KinticScan HCS Readers (Cellomics, Inc.). High-content screens can be performed on both live and fixed cells, preferably by using biosensors having multiple luminescent or fluorescent indicators, luminescent or fluorescent antibodies, biological ligands, or nucleic acid hybridization probes. The availability and use of luminescence or fluorescence-based reagents has advanced the development of both fixed and live cell high-content screens. In addition, advances in instrumentation to automatically extract multicolor, high-content information has recently made it possible to develop HCS into an automated tool (Taylor, et al., 1992).

In one embodiment, the detection and quantification can be performed using fixed cells. Fixed cell assays comprise, consist of, or consist essentially of an array of initially living cells in a microtiter plate format, which can be treated with various agents and doses being tested. The cells can then be fixed, labeled with specific reagents, and measured. No environmental control of the cells is required after fixation. Spatial information is acquired, but only at one time point. The availability of thousands of antibodies, ligands and nucleic acid hybridization probes that can be applied to cells makes this an attractive approach for many types of cell-based screens. The fixation and labeling steps can be automated, allowing efficient processing of assays.

In another embodiment, the detection and quantification is performed using live cells. Live cell assays are more sophisticated and powerful, since an array of living cells containing the desired reagents can be screened over time, as well as space. Environmental control of the cells (e.g., temperature, humidity, and carbon dioxide) is required during measurement, since the physiological health of the cells must be maintained for multiple luminescence or fluorescence measurements over time. Fluorescent or luminescent biosensors can be used to report changes in biochemical and molecular activities within cells (Giuliano et al., 1995; Mason, 1993).

In another preferred embodiment, a multiplexed HCS assay is used to quantify the effect of an agent of interest on cellular constituents or functions in the same cells, where at least two molecules that interact are introduced into a cell, the cellular constituent or function of interest is quantified, the cell is contacted by an agent of interest, after which the cellular constituent or function of interest is quantified, and the results are compared. A multiplexed assay also can be used to quantify the effect of the interaction between two molecules of interest on cellular constituents or functions in the same cells, where the cellular function of interest is quantified before and after the introduction of the molecules of interest concurrently to the cell. Multiplex HCS assays use HCS technology as described herein to conduct multi-parameter analyses that require only a single screening run. For example, multicolor fluorescence or luminescence can be used to detect and analyze multiple parameters at the same time. Preferably, the molecules that interact or molecules of interest are polypeptides. More preferably, at least one of the molecules is a biosensor. The cellular constituents or functions can be, for example, apoptosis; necrosis; cell cycle regulation; nuclear morphology; cellular DNA content; histone H3 phosphorylation levels; other kinase or phosphatase activities; transcription factor activation; tumor suppressor activation or induction; organellar functions including mitochondrial potential, peroxisome number and size, or endosomal pH; organization of the actin, microtubule, or intermediate filament cytoskeleton; receptor internalization or translocation; cell motility; protease activation; the heat shock response; exocytosis; endocytosis; cellular hypertrophy or other shape changes; and gene expression including coding and non-coding RNAs as well as proteins. Detection of various cellular constituents or functions of interest can be accomplished by using any luminescent or fluorescent reagent which allows detection of the constituent or function of interest, such as those described in DeBiasio et al., 1996, Giuliano et al., 1995, and Heim et al., 1996.

The inventive method can produce a database of information concerning the interaction of polypeptides with each other, cellular proteins or functions, and/or the effect of agents of interest on such interactions. This is most efficiently achieved wherein the inventive method is repeated or conducted with multiple iterations, for example using different biosensors, cells, and/or agents of interest. The invention provides such a database that comprises, consists of, or consists essentially of the results of the inventive methods described above. In addition, the database can comprise, consist of, or consist essentially of information on the interactions of molecules of interest and/or the effects of agents of interest on such interactions, and relate such information to cellular constituents and functions of interest. The database can, for example, be created using HCS technology or a multiplexed HCS assay, and compiling such information collected into a single record. Preferably, the database is constructed by screening a library for molecules or agents that have an effect on the interaction between two or more molecules of interest. More preferably, the database is constructed by screening a library for molecules or agents that disrupt the interaction between two or more cellular molecules.

The following examples further illustrate the invention but, of course, should not be construed as in any way limiting its scope.

### EXAMPLE 1

Many procedures discussed herein, such as luminescence and/or fluorescence tagging and detection, PCR, vector construction, including direct cloning techniques (including DNA extraction, isolation, restriction digestion, ligation, etc.), cell culture, transfection of cells, protein expression and purification, and HCS assays are techniques routinely performed by one of ordinary skill in the art (see generally Sambrook et al., Molecular Cloning, A Laboratory Manual, Cold Spring Harbor Laboratory, Cold Spring Harbor, N.Y. 1989).

This example demonstrates the construction and optimization of a modular biosensor to measure a specific protein-protein interaction in living cells. This biosensor is constructed to analyze the dynamic complex formation between the p53 tumor suppressor protein and its major intracellular binding partner, the HDM2 protein, which is the human homolog of MDM2. The approach outlined here can, however, be applied to the construction of other biosensors.

A eukaryotic expression plasmid that encodes a fusion protein consisting of an appropriate nuclear localization sequence (NLS), the p53 protein, and GFP is constructed. A separate expression vector will encode an appropriate nuclear export sequence (NES) joined with the coding sequence for HDM2. Co-transfection of the two plasmids into human A549 tumor cells expressing wild type p53 will produce cells with functional p53-HDM2 complexes distributed in both the cytoplasm and nucleus. Upon treatment with a disrupter of the p53-HDM2 interaction, the NLS-p53-GFP construct will redistribute with a nuclear bias. The cells will be treated with a bioactive green tea polyphenol, epigallocatechin-3-gallate (EGCG), and a cell-permeant p53-protein-derived peptide (Calbiochem) known to bind tightly (K_{d}=46 nM) to MDM2, the mouse homolog of HDM2 (Schon et al., 2002) as well as causing cytotoxicity over a period of several days in transformed cells (Kanovsky et al., 2001). The cells will also be treated with a new small molecule inhibitor of the p53-HDM2 interaction, Nutlin-3 (IC₅₀=90 nM) (Vassilev et al., 2004), that has recently become commercially available (Alexis Biochemicals). These inhibitory compounds will act as competitive inhibitors of the p53-HDM2 interaction, thus inducing a biased shuttling of the NLS-p53-GFP biosensor into the nucleus. Biosensor pairs containing the combination of NES and NLS that show the greatest redistribution after treatment with the inhibitory compounds, quantified using the multiplexed HCS assay described above, will be used in a kinetic HCS assay to further characterize the response of the system to determine inhibitory compound concentrations and treatment times that reproducibly induce the largest responses.

Preparation of cells expressing NLS-p53-GFP and NES-HDM2. To produce cells expressing biosensors, the strategy for the transient double transfection of mammalian cells with p53-GFP and HDM2 described by Boyd et al. (2000) will be followed. Briefly, cDNA encoding the appropriate NLS and wild type p53 will be cloned upstream of GFP in a pEGFP/N1 vector (Clontech). The necessary amplification will be carried out using PCR with primers encoding PstI and BamHI endonuclease sites. HDM2 cDNA (Chen et al., 1994) will be fused with an appropriate NES and inserted into a pcDNA3.1 expression vector (Invitrogen), which, like the pEGFP/N1 vector, is under the control of the CMV promoter. A549 cells will be grown in log phase to a density of 2.5×10⁺⁶ cells per T-25 flask. The cells will be transfected with a mixture of expression plasmids encoding the NLS-p53-GFP and NES-HDM2 at 2 μg per T-25 flask, using Lipofectamine 2000 transfection reagent (Invitrogen). After an 18-24 hour incubation, the transfected cells will be trypsinized and plated at 6000-8000 cells per well in a collagen I coated 384-well microplate (Falcon #3962). Some of the cells will be labeled at this point using an antibody against HDM2 (Upstate) to ensure that both the NLS-p53-GFP and NES-HDM2 are expressed in the transfected cells. Cells at this stage are ready for use in either live cell kinetic or fixed end point HCS assays.

Selection of appropriate NES and NLS structures for biosensor construction. Each new positional biosensor will have specific requirements for the nuclear-cytoplasmic shuttling components (Giuliano et al., 2003 a), but an automated processes that permits the parallel development and testing of multiple biosensors has been developed. The first design of the p53-HDM2 biosensor will involve choosing combinations of NES and NLS structures that vary in transport activity based on detailed reports correlating particular localization sequences with their transport activity. Combinations of the sequences SEQ ID NO:1-6 will be used to prepare the initial biosensors. Kinetic and fixed end point HCS assays provide the ideal means to quantify the usefulness of each signaling sequence combination. Use of multiple copies of the same localization sequence could potentially be used to further tune biosensor localization.

Characterization of response times of the biological assay system using live cell kinetic measurements of NLS-p53-GFP nuclear translocation. To develop the p53-HDM2 biosensor and optimize its incorporation into the recently developed multiplexed fixed end point HCS assay, the time course of the p53-HDM2 complex disruption will be measured in a live cell kinetic HCS mode (Abraham et al., 2004). Cells expressing NLS-p53-GFP and NES-HDM2 will be treated with different concentrations of inhibitory compounds and the time course of the intracellular redistribution of the NLS-p53-GFP will be measured over a time period of 24 hours after treatment using a KineticScan HCS reader (Cellomics, Inc.). This instrument automatically makes multiple measurements of the cytoplasm-nuclear distribution of NLS-p53-GFP while maintaining the health of the cells (Abraham et al., 2004). The kinetic analysis will provide several benefits. First, the rate and extent of biosensor expression will be calculated automatically for each cell during the kinetic analysis. These data will be used to optimize the transfection efficiency. Second, the kinetic analysis will allow a direct assessment of the effect that biosensor expression itself has on multiple aspects of cell health. Finally, the quantitative results of these experiments (e.g., concentrations of inhibitory compounds that disrupt the p53-HDM2 interaction over a specified time period) will facilitate the incorporation of the biosensor into the multiplexed HCS assay. Compound library screening then can proceed directly.

Identification of possible non-specific or “other” protein interactions. If experimental cell treatments induce measurable cytoplasm to nuclear translocation of the biosensors, then confirmatory assays will be performed to test for non-specific or “other” protein interactions. In the case of the p53-HDM2 biosensor, cells will be transfected to express only the NLS-p53-GFP biosensor. The unpaired NLS-p53-GFP biosensor will be predominately distributed in the cell nucleus. If the experimental treatment induces a non-specific interaction between p53 and another protein, or if the NLS-p53-GFP interacts strongly with another (specific or non-specific) endogenous cytoplasmic protein, then the likely result will be at least a partial redistribution of the NLS-p53-GFP into the cytoplasm. In addition, cells will also be transfected to express only the NES-HDM2 biosensor. If the inhibitory compounds induce a non-specific or another specific interaction between HDM2 and another protein, then the resulting redistribution of the NES-HDM2 biosensor using an antibody against the epitope tag will be measured with the multiplexed HCS assay. Protein-protein interactions other than the targeted ones detected might involve other relevant protein binding partners. Thus, as these new binding partners are identified, additional biosensors will be designed to measure their interactions and the effects experimental treatments have on them. The growing interactome will be continually mined to identify new potential binding partners. New biosensors will then be constructed to test these predictions. Furthermore, multiplex positional biosensors can be created by using distinct compartments to target the binding partner constructs (e.g., cytoplasm-nucleus, cytoplasm-plasma membrane, etc.) with a single channel of fluorescence or luminescence.

Alternative strategy for the transient transfection approach. Double transient transfection of p53-GFP and HDM2 approach has been used successfully (Boyd et al., 2000) and the gating capabilities of HCS have made it possible to use transiently transfected cell populations in large scale screens. However, in some instances multiple transient transfection may not be not sufficient. Thus, an alternative strategy for the preparation of cellular reagents can be pursued. Clonal cell lines stably overexpressing both components of the biosensor will be prepared using automated liquid handling robots and HCS analyses. These cell lines can be a panel consisting of multiple tumor types comprised of populations of cells substantially uniformly expressing the biosensor components throughout at least 10 passages. While it is believed that the transient transfection approach will be sufficient for the proof of principle experiments proposed here, this alternative strategy for cell-based reagent preparation will be exercised when necessary or required for commercialization.

Alternative tumor cell types. Human tumor cell line A549 has previously shown utility in HCS assays and expresses wild type p53 protein, thus it will be the initial cell line used. If this cell line is inadequate, however, due to low transfection efficiency, poor kinetic response, severe toxicity, or other factors, additional cell lines will be tested. The first alternative cell lines will be those that are p53 protein null in case the background expression of p53 protein is the cause of a poor response. These alternative human tumor cell lines will be MDA-MB-435 (breast carcinoma), H1299 (non-small cell lung cancer), and Saos-2 (osteosarcoma).

### EXAMPLE 2

This example demonstrates the construction and optimization of a modular biosensor to measure classes of protein binding domain interactions in living cells. This biosensor is constructed to analyze the dynamic complex formation between the src-homology domain 2 (SH2) and its target molecules within living human cells. The approach outlined here can, however, be applied to the construction of other biosensors.

To initiate the design of fluorescent protein biosensors that measure how chemical compounds modulate the communications between protein interaction domains and their targets, a biosensor will be constructed based on the interaction of an SH2 domain and its tyrosine-phosphorylated protein targets. It has been estimated that more than 100 SH2 protein interaction domains are encoded by the human genome (Pawson et al., 2003). Furthermore, a single protein containing an SH2 domain (˜100 amino acids) can itself potentially interact with many other proteins, including membrane bound receptors, soluble enzymes, and cytoskeletal proteins. The human cell maintains thousands of SH2-dependent protein-protein interactions thus forming the foundation of a crucial signaling nexus and an optimal target for drug modulation. A biosensor will be designed to measure the dynamic interactions between an SH2 domain, isolated from the c-src kinase, and its target molecules within living human cells. For example, the SH2 domain biosensor can be used to measure the stimulation of a receptor tyrosine kinase. Ligand binding by the receptor initiates a cascade of processes that result in the appearance of a large number of SH2 domain binding sites. The fluorescent or luminescent SH2 domain biosensor responds by binding to these newly available sites in the cytoplasm, shifting the equilibrium distribution of the biosensor toward the cytoplasmic compartment. Thus, the ratio between the cytoplasmic concentration of the biosensor and its nuclear concentration will become significantly larger upon receptor stimulation. An HCS assay can be used to quantify changes in the cytoplasm-nuclear distribution ratio, permitting the effects of chemical compounds to be screened on a large scale. This approach will produce a qualified set of compounds with the capability of modulating molecular processes under the regulation of SH2 protein interaction domains.

A eukaryotic expression plasmid encoding a single biosensor consisting of an NLS and an NES, an SH2 protein domain, and a GFP will be transfected into human A431 tumor cells, which constitutively over express epidermal growth factor receptor (EGFR). The A431 cells will be stimulated with EGF, and in some cases, also with EGCG. The intracellular redistribution of the biosensors between the nucleus and the cytoplasm will then be measured kinetically to determine the time course of the redistribution. The biosensor containing the NES and NLS combination that shows the greatest redistribution after stimulation with EGF will be used in the multiplexed HCS assay to further characterize the response of the system to determine EGF concentrations that reproducibly induce the largest measurable responses.

Selection of appropriate NES and NLS structures for biosensor construction. Combinations of the 6 localization peptide sequences SEQ ID NO: 1-6 will be utilized to prepare 4 initial biosensors. HCS assays will provide the ideal means to quantify the usefulness of each signaling sequence combination. Thus, the 4 fusion proteins will comprise molecular components similar to those described in Example 1. Nucleic acids encoding NLS and NES combinations will flank sequences encoding the SH2 and GFP domains. The SH2 domain will be encoded using the pp 60Src SH2 domain (corresponding to amino acids 142-251), which has already been used to produce a GFP chimera that retains activity when expressed in living cells (Kirchner et al., 2003).

Characterization of response times of the biological assay system using live cell kinetic measurements of SH2 protein domain activation will be performed. As described in Example 1, the SH2 biosensor will be validated by measuring the time course of ligand-induced redistribution of the biosensor in a live cell kinetic HCS mode. Briefly, cells expressing the SH2 biosensor variants will be treated with 100 ng/ml EGF and the time course of intracellular redistribution of each of the biosensor types will be measured every minute over a time course of 1 hour (Yamazaki et al., 2002). The quantitative results of these experiments will facilitate the incorporation of the biosensor into the multiplexed HCS assay. Compound screening will proceed immediately thereafter.

### EXAMPLE 3

This example demonstrates the integration of the biosensors into a validated, multiplexed HCS assay that defines the effects compounds have on molecular interactions while placing them in the context of a multi-featured phenotype. Measurements of nuclear morphology, cellular DNA content, microtubule-stability, and histone H3 phosphorylation level can be made. The assay will be demonstrated by screening a library of 500-1000 compounds for modulators of target protein-protein interactions.

The biosensors will be integrated into the multiplexed HCS assay where the molecular processes measured have physiological connections to the biological activities measured with the biosensors. When the biosensors are incorporated, the assay will be 4-color. In addition, the assay will be validated using a published statistical analysis method based on the Kolmogorov-Smirnov goodness of fit test (Giuliano et al., 2004). Hoechst 33342 is used for DNA content and nuclear morphology measurements, a mouse anti-β-tubulin primary antibody is used to assess remaining cellular tubulin after detergent extraction, and anti-phospho-histone H3 primary antibody will be used to measure the phosphorylation level of histone H3.

Cell transfection and drug treatment. Expression vectors encoding biosensors will be transfected into cells as described in Example 1. For drug treatment, transfected cells will be plated at a density of 6000-8000 cells per well in 384-well microplates. Cells will be exposed to drugs for 24 hours after the addition of concentrated stocks of all drugs to microplates using an automated liquid handling system (Biomek 2000; Beckman-Coulter, Inc., Fullerton, Calif.).

Immunofluorescence labeling using automated liquid handling. After drug treatment, the cells are fixed and their nuclei labeled (Giuliano et al., 2004). Next, 0.5% (w/w) Triton X-100 is added and incubated for 5 minutes at room temperature to detergent extract a fraction of the soluble cellular components including destabilized tubulin. The wells are washed with HBSS followed by the addition of a primary antibody solution containing mouse anti-α-tubulin and rabbit anti-phospho-histone H3. After a 1 hour incubation at room temperature, the microplate wells are washed with HBSS followed by the addition of a secondary antibody solution containing Cy3-labeled donkey anti-mouse and Cy5-labeled donkey anti-rabbit antibodies. After 1 hour incubation at room temperature, the microplate wells are washed with HBSS and stored until HCS.

HCS process. HCS is performed with an ArrayScan® HCS Reader with the Compartmental Analysis BioApplication Software coupled to Cellomics® Store and the vHCS™ Discovery Toolbox (Cellomics, Inc.; Pittsburgh, Pa.). The instrument is used to scan multiple optical fields, each with multiplexed fluorescence or luminescence, within the wells of the microplate. The BioApplication software produces more than thirty numerical feature values such as subcellular object intensities, shapes, and location for each cell within an optical field. Some aspects of these algorithms have been described (Abraham et al., 2004). The number of cells measured per well is determined by statistical requirements.

Statistical analysis and screen validation. To determine the significance of the cellular population response, a Kolmogorov-Smimov goodness of fit analysis is used (KS statistic). Briefly, the sample size determines a critical KS statistic value, which when corrected for variation due to biological heterogeneity, is sufficient to distinguish a significant biological effect of drug treatment relative to a control sample treated with vehicle only (Giuliano et al., 2004). Therefore, the KS value will be the determining factor in deciding if compounds produce a “hit” against one or more of the multiplexed targets, and in providing a measure of activity that can be used to rank compounds relative to each other.

Experimental and biological variability. The second component of assay validation involves the inherent variability of the cell-based experimental system on a day-to-day basis. Besides the experimental variability contributed by instrumentation such as liquid handling robots and microplate readers, the biological variability of the target cells and several of the immunoreagents dominate the reproducibility of the entire screening platform. To address biological variability in a practical way, strict standard operating procedures will be maintained with respect to cell handling and reagent procurement and preparation. An assay will therefore be considered validated if it returns results with the same level of statistical significance, as described above, after three separate trials.

Alternative HCS assay parameters. The HCS assay parameters chosen for the initial design complement the cellular processes that will be measured with the biosenors described in Examples 1 and 2. Nevertheless, the inherent flexibility of the assay system allows for facile swapping of other reagents that report on different sets of physiological parameters. The multiplexed HCS assay may be designed with a bias toward transcription factor activation to highlight the connections between biosensor activity and gene expression. The alternative HCS assay could involve measurement of the activation of NF-κB, ATF-2, NFAT, or one or more of the STAT family members of transcription factors. Furthermore, biosensor activity could be measured in combination with the activation of three stress kinases JNK, p38 MAPK, and ERK.

All references, including publications, patent applications, and patents, cited herein, including the following list, are hereby incorporated by reference to the same extent as if each reference were individually and specifically indicated to be incorporated by reference and were set forth in its entirety herein.
i. Abraham, V. C., D. L. Taylor, and J. R. Haskins. 2004. High content screening applied to large-scale cell biology. Trends In Biotechnology. 22:15-22.
ii. Arkin, M. R., and J. A. Wells. 2004. Small-molecule inhibitors of protein-protein interactions: progressing towards the dream. Nat Rev Drug Discov. 3:301-17.
iii. Bantscheff, M., B. Dumpelfeld, and B. Kuster. 2004. Femtomol sensitivity post-digest (18)O labeling for relative quantification of differential protein complex composition. Rapid Commun Mass Spectrom. 18:869-76.
iv. Bastiaens et al. 1999. Fluorescence lifetime imaging microscopy: spatial resolution of biochemical processes in the cell. Trends Cell Biology, 9(2):48-52.
v. Bauer, A., and B. Kuster. 2003. Affinity purification-mass spectrometry. Eur J Biochem. 270:570-8.
vi. Bork, P., L. J. Jensen, C. Von Mering, A. K. Ramani, I. Lee, and E. M. Marcotte. 2004. Protein interaction networks from yeast to human. Curr Opin Struct Biol. 14:292-9.
vii. Boyd, S. D., K. Y. Tsai, and T. Jacks. 2000. An intact HD RING-finger domain is required for nuclear exclusion of p53. Nat Cell Biol. 2:563-8.
viii. Chapman, T. 2004. Drug discovery: the leading edge. Nature. 430:109-15.
ix. Chen, C. Y., J. D. Oliner, Q. Zhan, A. J. Fornace, Jr., B. Vogelstein, and M. B. Kastan. 1994. Interactions between p53 and MDM2 in a mammalian cell cycle checkpoint pathway. Proc Natl Acad Sci USA. 91:2684-8.
x. Chen, Y., J. D. Mills, and A. Periasamy. 2003. Protein localization in living cells and tissues using FRET and FLIM. Differentiation. 71:528-41.
xi. Chene, P. 2004. Inhibition of the p53-MDM2 interaction: targeting a protein-protein interface. Mol Cancer Res. 2:20-8.
xii. Colland, F., X. Jacq, V. Trouplin, C. Mougin, C. Groizeleau, A. Hamburger, A. Meil, J. Wojcik, P. Legrain, and J. M. Gauthier. 2004. Functional proteomics mapping of a human signaling pathway. Genome Res. 14:1324-32.
xiii. DeBiasio et al., 1996. Myosin II transport, organization, and phosphorylation: evidence for cortical flow/solation-contraction coupling during cytokinesis and cell locomotion. Mol. Biol. Cell. 7(8):1259-82.
xiv. Farkas et al. 1993. Multimode light microscopy and the dynamics of molecules, cells, and tissues. Ann. Rev. Physiol. 55:785.
xv. Farr-Jones, S., and S. J. Fox. 2004. High-content screening for drug discovery and target validation. Decision Resources, Waltham, Mass.
xvi. Filhol, O., A. Nueda, V. Martel, D. Gerber-Scokaert, M. J. Benitez, C. Souchier, Y. Saoudi, and C. Cochet. 2003. Live-cell fluorescence imaging reveals the dynamics of protein kinase CK2 individual subunits. Mol Cell Biol. 23:975-87.
xvii. Frost, and Sullivan. 2003. World functional proteomics markets. Frost & Sullivan Research Report.
xviii. Gadek, T. R., and J. B. Nicholas. 2003. Small molecule antagonists of proteins. Biochem Pharmacol. 65:1-8.
xix. Giot, L., J. S. Bader, C. Brouwer, A. Chaudhuri, B. Kuang, Y. Li, Y. L. Hao, C. E. Ooi, B. Godwin, E. Vitols, G. Vijayadamodar, P. Pochart, H. Machineni, M. Welsh, Y. Kong, B. Zerhusen, R. Malcolm, Z. Varrone, A. Collis, M. Minto, S. Burgess, L. McDaniel, E. Stimpson, F. Spriggs, J. Williams, K. Neurath, N. Ioime, M. Agee, E. Voss, K. Furtak, R. Renzulli, N. Aanensen, S. Carrolla, E. Bickelhaupt, Y. Lazovatsky, A. DaSilva, J. Zhong, C. A. Stanyon, R. L. Finley, Jr., K. P. White, M. Braverman, T. Jarvie, S. Gold, M. Leach, J. Knight, R. A. Shimkets, M. P. McKenna, J. Chant, and J. M. Rothberg. 2003. A protein interaction map of Drosophila melanogaster. Science. 302:1727-36.
xx. Giuliano, K. A., Y. T. Chen, and J. R. Haskins. 2003a. Positional biosensors: A new tool for high-content screening. Modern Drug Discovery. August:33-37.
xxi. Giuliano, K. A., Y. T. Chen, and D. L. Taylor. 2004. High-content screening with siRNA optimizes a cell biological approach to drug discovery: Defining the role of p53 activation in the cellular response to anticancer drugs. J. Biomol. Screening. 9:557-568.
xxii. Giuliano, K. A., R. L. DeBiasio, R. T. Dunlay, A. Gough, J. M. Volosky, J. Zock, G. N. Pavlakis, and D. L. Taylor. 1997. High-content screening: A new approach to easing key bottlenecks in the drug discovery process. Journal of Biomolecular Screening. 2:249-259.
xxiii. Giuliano, K. A., J. R. Haskins, and D. L. Taylor. 2003b. Advances in high content screening for drug discovery. ASSAY and Drug Development Technologies. 1:565-577.
xxiv. Giuliano, K. A., P. L. Post, K. M. Hahn, and D. L. Taylor. 1995. Fluorescent protein biosensors: Measurement of molecular dynamics in living cells. Annual Review of Biophysics and Biomolecular Structure. 24:405-434.
xxv. Giuliano, K. A., and D. L. Taylor. 1995. Measurement and manipulation of cytoskeletal dynamics in living cells. Current Opinion in Cell Biology. 7:4-12.
xxvi. Giuliano, K. A., and D. L. Taylor. 1998. Fluorescent-protein biosensors: New tools for drug discovery. Trends In Biotechnology. 16:135-140.
xxvii. Gough, A. H., and D. L. Taylor. 1993. Fluorescence anisotropy imaging microscopy maps calmodulin binding during cellular contraction and locomotion. Journal of Cell Biology. 121:1095-1107.
xxviii. Heim et al., 1996. Engineering green fluorescent protein for improved brightness, longer wavelengths and fluorescence resonance energy transfer. Curr. Biol. 6(2): 178-82.
xxix. Henderson, B. R., and A. Eleftheriou. 2000. A comparison of the activity, sequence specificity, and CRM1-dependence of different nuclear export signals. Exp Cell Res. 256:213-24.
xxx. Hodel, M. R., A. H. Corbett, and A. E. Hodel. 2001. Dissection of a nuclear localization signal. J Biol Chem. 276:1317-25.
xxxi. Ito, T., T. Chiba, R. Ozawa, M. Yoshida, M. Hattori, and Y. Sakaki. 2001. A comprehensive two-hybrid analysis to explore the yeast protein interactome. Proc Natl Acad Sci USA. 98:4569-74.
xxxii. Kanovsky, M., A. Raffo, L. Drew, R. Rosal, T. Do, F. K. Friedman, P. Rubinstein, J. Visser, R. Robinson, P. W. Brandt-Rauf, J. Michl, R. L. Fine, and M. R. Pincus. 2001. Peptides from the amino terminal mdm-2-binding domain of p53, designed from conformational analysis, are selectively cytotoxic to transformed cells. Proc Natl Acad Sci USA. 98:12438-43.
xxxiii. Kirchner, J., Z. Kam, G. Tzur, A. D. Bershadsky, and B. Geiger. 2003. Live-cell monitoring of tyrosine phosphorylation in focal adhesions following microtubule disruption. J Cell Sci. 116:975-86.
xxxiv. Kussie et al. 1996, Structure of the MDM2 oncoprotein bound to the p53 tumor suppressor transactivation domain. Science. November 8; 274(5289):948-53.
xxxv. Li, S., C. M. Armstrong, N. Bertin, H. Ge, S. Milstein, M. Boxem, P. O. Vidalain, J. D. Han, A. Chesneau, T. Hao, D. S. Goldberg, N. Li, M. Martinez, J. F. Rual, P. Lamesch, L. Xu, M. Tewari, S. L. Wong, L. V. Zhang, G. F. Berriz, L. Jacotot, P. Vaglio, J. Reboul, T. Hirozane-Kishikawa, Q. Li, H. W. Gabel, A. Elewa, B. Baumgartner, D. J. Rose, H. Yu, S. Bosak, R. Sequerra, A. Fraser, S. E. Mango, W. M. Saxton, S. Strome, S. Van Den Heuvel, F. Piano, J. Vandenhaute, C. Sardet, M. Gerstein, L. Doucette-Stamm, K. C. Gunsalus, J. W. Harper, M. E. Cusick, F. P. Roth, D. E. Hill, and M. Vidal. 2004. A map of the interactome network of the metazoan C. elegans. Science. 303:540-3.
xxxvi. Mason (ed.) 1993. Fluorescent and Luminescent Probes for Biological Activity, 124-132.
xxxvii. Michnick, S. W. 2004. Proteomics in living cells. Drug Discov Today. 9:262-7.
xxxviii. Miyawaki, A., A. Sawano, and T. Kogure. 2003. Lighting up cells: labelling proteins with fluorophores. Nat Cell Biol. Suppl:S1-7.
xxxix. Nagar, B., O. Hantschel, M. A. Young, K. Scheffzek, D. Veach, W. Bornmann, B. Clarkson, G. Superti-Furga, and J. Kuriyan. 2003. Structural basis for the autoinhibition of c-Abl tyrosine kinase. Cell. 112:859-71.
xl. Nelson, G., L. Paraoan, D. G. Spiller, G. J. Wilde, M. A. Browne, P. K. Djali, J. F. Unitt, E. Sullivan, E. Floettmann, and M. R. White. 2002. Multi-parameter analysis of the kinetics of NF-kappaB signalling and transcription in single living cells. J Cell Sci. 115:1137-48.
xli. Offterdinger, M., V. Georget, A. Girod, and P. I. Bastiaens. 2004. Imaging phosphorylation dynamics of the EGF receptor. J Biol Chem. 279:36972-36981.
xlii. Oren, M., A. Damalas, T. Gottlieb, D. Michael, J. Taplick, J. F. Leal, R. Maya, M. Moas, R. Seger, Y. Taya, and A. Ben-Ze'ev. 2002. Regulation of p53: intricate loops and delicate balances. Biochem Pharmacol. 64:865-71.
xliii. Pawson, T. 2003. Organization of cell-regulatory systems through modular-protein-interaction domains. Philos Transact Ser A Math Phys Eng Sci. 361:1251-62.
xliv. Pawson, T., and P. Nash. 2003. Assembly of cell regulatory systems through protein interaction domains. Science. 300:445-52.
xlv. Perlman, Z. E., M. D. Slack, Y. Feng, T. J. Mitchison, L. F. Wu, and S. J. Altschuler. 2004. Multidimensional drug profiling by automated microscopy. Science. 306:1194-8.
xlvi. Rizzo, M. A., and D. W. Piston. 2005. High-Contrast Imaging of Fluorescent Protein FRET by Fluorescence Polarization Microscopy. Biophys J. 88:L14-6.
xlvii. Rubinfeld et al., J. Biol. Chem., 274, 30349-52 (1999)
xlviii. Sah, J. F., S. Balasubramanian, R. L. Eckert, and E. A. Rorke. 2004. Epigallocatechin-3-gallate Inhibits Epidermal Growth Factor Receptor Signaling Pathway: EVIDENCE FOR DIRECT INHIBITION OF ERK1/2 AND AKT KINASES. J Biol Chem. 279:12755-62.
xlix. Schon, O., A. Friedler, M. Bycroft, S. M. Freund, and A. R. Fersht. 2002. Molecular mechanism of the interaction between MDM2 and p53. J Mol Biol. 323:491-501.
l. Taylor, D. L., and Y. L. Wang. 1978. Molecular cytochemistry: Incorporation of fluorescently labeled actin into cells. Proceedings of the National Academy of Sciences. 75:857-861.
li. Taylor, D. L., E. S. Woo, and K. A. Giuliano. 2001. Real-time molecular and cellular analysis: the new frontier of drug discovery. Curr Opin Biotechnol. 12:75-81.
lii. Taylor, et al. 1992. The New Technology of Light Microscopy. American Scientist 80:322-335.
liii. Thomas, N., and I. D. Goodyer. 2003. Stealth sensors: real-time monitoring of the cell cycle. Targets. 2:26-33.
liv. Toogood, P. L. 2002. Inhibition of protein-protein association by small molecules: approaches and progress. J Med Chem. 45:1543-58.
lv. Uetz, P., L. Giot, G. Cagney, T. A. Mansfield, R. S. Judson, J. R. Knight, D. Lockshon, V. Narayan, M. Srinivasan, P. Pochart, A. Qureshi-Emili, Y. Li, B. Godwin, D. Conover, T. Kalbfleisch, G. Vijayadamodar, M. Yang, M. Johnston, S. Fields, and J. M. Rothberg. 2000. A comprehensive analysis of protein-protein interactions in Saccharomyces cerevisiae [see comments]. Nature. 403:623-7.
lvi. Vassilev, L. T., B. T. Vu, B. Graves, D. Carvajal, F. Podlaski, Z. Filipovic, N. Kong, U. Kammlott, C. Lukacs, C. Klein, N. Fotouhi, and E. A. Liu. 2004. In vivo activation of the p53 pathway by small-molecule antagonists of MDM2. Science. 303:844-8.
lvii. Yamazaki, T., K. Zaal, D. Hailey, J. Presley, J. Lippincott-Schwartz, and L. E. Samelson. 2002. Role of Grb2 in EGF-stimulated EGFR internalization. J Cell Sci. 115:1791-802.
lviii. Yeh, R. H., X. Yan, M. Cammer, A. R. Bresnick, and D. S. Lawrence. 2002. Real time visualization of protein kinase activity in living cells. J Biol Chem. 277:11527-32.
lix. Zacharias, D. A., G. S. Baird, and R. Y. Tsien. 2000. Recent advances in technology for measuring and manipulating cell signals. Curr Opin Neurobiol. 10:416-21.
lx. Zhu, H., M. Bilgin, and M. Snyder. 2003. Proteomics. Annu Rev Biochem. 72:783-812.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method for detecting the effect of an agent of interest on the interaction between two or more polypeptides, the method comprising:
a. introducing two or more polypeptides (e.g., a first polypeptide and a second polypeptide), into a cell under conditions wherein said polypeptides interact with each other, endogenous proteins, or a combination thereof, and wherein at least said first polypeptide is a biosensor comprising an interaction domain that interacts with one or more polypeptides, endogenous proteins, or a combination thereof and further comprising a reporter domain, and quantifying the interaction between one or more of said polypeptides, one or more of said endogenous proteins, or combinations thereof,
b. contacting said cell with an agent of interest and quantifying the interaction between said polypeptides, endogenous proteins, or a combination thereof, and
c. comparing the result of step (a) to that of step (b).

2. The method of claim 1, wherein said second polypeptide also is a biosensor comprising an interaction domain that interacts with polypeptides, endogenous proteins, or a combination thereof and further comprising a reporter domain.

3. The method of claim 1, wherein a reporter domain comprises a luminescent or fluorescent moiety.

4. The method of claim 2, wherein a reporter domain comprises a luminescent or fluorescent moiety.

5. The method of claim 3 where the luminescent or fluorescent moiety is GFP.

6. The method of claim 3, wherein the interaction between said polypeptides is evaluated by assessing a fluorescence of luminescence signal change.

7. The method of claim 4, wherein the interaction between said polypeptides is evaluated by assessing a fluorescence or luminescence signal change.

8. A method for detecting the effect of an agent of interest on the interaction between two or more polypeptides, the method comprising:
a. introducing two or more polypeptides (e.g., a first polypeptide and a second polypeptide), into a cell under conditions wherein said polypeptides interact with each other, endogenous proteins, or a combination thereof, and wherein at least said first polypeptide is a biosensor comprising a reporter comprising a luminescent or fluorescent moiety, and quantifying the interaction between one or more of said polypeptides, one or more of said endogenous proteins, or combinations thereof by assessing a reversible fluorescence or luminescence signal change,
b. contacting said cell with an agent of interest and quantifying the interaction between one or more of said polypeptides, one or more of said endogenous proteins, or combinations thereof by assessing a fluorescence or luminescence signal change, and
c. comparing the result of step (a) to that of step (b).

9. The method of claim 8, wherein said second polypeptide also is a biosensor comprising a reporter comprising a luminescent or fluorescent moiety.

10. The method of claim 9 where the luminescent or fluorescent moiety is GFP.

11. The method of claim 8, wherein said first and/or said second polypeptide further comprises an interaction domain that interacts with one or more of said polypeptides, one or more of said endogenous proteins, or combinations thereof.

12. The method of claim 1, wherein said agent is a chemical, physical stimulus, environmental stimulus, electrical stimulus, or radiation.

13. The method of claim 8, wherein said agent is a chemical, physical stimulus, environmental stimulus, electrical stimulus, or radiation.

14. A method for quantifying the interaction between at least two molecules of interest, the method comprising:
a. introducing each molecule of interest individually into a cell, wherein at least one of said molecules of interest comprises a reporter comprising a luminescent or fluorescent moiety, and quantifying the level of luminescence or fluorescence,
b. introducing said molecules of interest into a cell concurrently and quantifying the level of luminescence or fluorescence, and
c. comparing the result of step (a) to that of step (b).

15. The method of claim 14, where one of the molecules of interest is SH2.

16. The method of claim 14, wherein a molecule of interest is a polypeptide.

17. The method of claim 16, wherein the polypeptide further comprises an interaction domain that interacts with one or more of said polypeptides, one or more of said endogenous proteins, or combinations thereof.

18. The method of claim 6, wherein the interaction is quantified using fluorescence resonance energy transfer, fluorescence anisotropy, rotational difference, fluorescence lifetime change, fluorescence solvent sensitivity, and fluorescence quenching.

19. The method of claim 1, where the interaction is automatically quantified by a device.

20. The method of claim 19, wherein the device provides an array of locations which contain multiple cells, scans multiple cells in each of the locations containing cells to obtain luminescent or fluorescent signals from at least one luminescent or fluorescent reporter polypeptide within the cells; measuring a luminescence or fluorescence intensity of the luminescent or fluorescent signals from at least one luminescent or fluorescent reporter polypeptide within a specific location in the cells; and automatically calculating changes induced by the polypeptide of interest in one or more of the following:
a. a ratio of luminescent or fluorescent signal intensity from at least one luminescent or fluorescent reporter polypeptide in a specific location in the cells to luminescent or fluorescent signal intensity from at least one luminescent or fluorescent reporter polypeptide in a different specific location in the cells; and
b. a difference between luminescent or fluorescent signal intensity from the at least one luminescent or fluorescent reporter polypeptide in a specific location in the cells and luminescent or fluorescent signal intensity from at least one luminescent or fluorescent reporter polypeptide in a different specific location in the cells wherein the changes induced by the agent of interest indicate an effect of the agent of interest on the localization of the polypeptide from a first location in the cells to a second specific location in the cells.

21. A method for detecting the effect of an agent of interest on cellular constituents or functions in the same cells, the method comprising:
a. introducing two or more molecules that interact into a cell and quantifying the cellular constituent or function of interest,
b. contacting said cell with an agent of interest and quantifying the cellular constituent or function of interest, and
c. comparing the result of step (a) to that of step (b).

22. The method of claim 21, wherein a molecule is a polypeptide.

23. A method for quantifying the effect of the interaction between two molecules of interest on cellular constituents or functions in the same cells, the method comprising:
a. introducing each molecule of interest individually into a cell and quantifying the cellular constituent or function of interest,
b. introducing said molecules of interest into said cell concurrently and quantifying the cellular constituent or function of interest, and
c. comparing the result of step (a) to that of step (b).

24. The method of claim 23, wherein a molecule is a polypeptide.

25. The method of claim 21, wherein the cellular constituents or functions of interest are selected from the group consisting of apoptosis; necrosis; cell cycle regulation; nuclear morphology; cellular DNA content; histone H3 phosphorylation levels; other kinase or phosphatase activities; transcription factor activation; tumor suppressor activation or induction; organellar functions including mitochondrial potential, peroxisome number and size, or endosomal pH; organization of the actin, microtubule, or intermediate filament cytoskeleton; receptor internalization or translocation; cell motility; protease activation; the heat shock response; exocytosis; endocytosis; cellular hypertrophy or other shape changes; and gene expression including coding and non-coding RNAs as well as proteins.

26. The method of claim 1, wherein one or more of said polypeptides comprises a localization domain which causes said polypeptide to be expressed with a specific localization pattern in the cell, and the agent of interest disrupts said localization pattern.

27. The method of claim 26, where one or more of said polypeptides contains more than one localization domain.

28. The method of claim 26, where said localization domain is selected from the group consisting of a NES and NLS.

29. The method of claim 26, wherein the localization domain comprises or consists essentially of an amino acid sequence selected from the group consisting of

| | |
|---|---|
| KRTADGSEFESPKKARKVE, | (SEQ ID NO:1) |
| QQMGRGSEFEPAAKRAKLDE, | (SEQ ID NO:2) |
| QQMGRGSEFESPKKARKVE, | (SEQ ID NO:3) |
| NSNELALKLAGLDINKTE, | (SEQ ID NO:4) |
| HAEKVAEKLEALSVKEET, | (SEQ ID NO:5) |
| and | |
| PSTRIQQQLGQLTLENLQ. | (SEQ ID NO:6) |

30. The method of claim 1, wherein at least one of said polypeptides comprises a protein, protein fragment, or protein interaction domain.

31. The method of claim 1, wherein at least one of said polypeptides comprises a protein and is expressed within in the cell under the transcriptional control of an inducible promoter.

32. The method of claim 1, wherein at least one of said polypeptides is produced outside of the cell and then introduced into the cell.

33. The method of claim 1, wherein at least one of said polypeptides is exogenous to said cell.

34. The method of claim 1, wherein at least one of said polypeptides is endogenous to said cell.

35. The method of claim 1, wherein at least two of said polypeptides are p53 and HDM2.

36. The method of claim 1, wherein at least one of said polypeptides has the sequence SEQ ID NO: 7.

37. The method of claim 1, wherein at least one of said polypeptides has the sequence SEQ ID NO: 8.

38. The method of claim 1, wherein at least one of said polypeptides has the sequence SEQ ID NO: 9.

39. The method of claim 1, wherein at least one of said polypeptides has the sequence SEQ ID NO: 10.

40. The method of claim 1, wherein at least one of said polypeptides has the sequence SEQ ID NO: 11.

41. The method of claim 1, wherein at least one of said polypeptides has the sequence SEQ ID NO: 12.

42. A method of High Content Screening (HCS) comprising the method of claim 1, wherein the measurements are taken using HCS techniques.

43. The method of claim 1, wherein said method is repeated using different molecules and/or agents of interest, whereby the results of said repeated assays are compiled into a database of quantified comparisons.

44. A database created using the method of claim 43.

45. The database of claim 44, comprising information on the interactions of molecules of interest with other molecules or agents and relating said interactions to additional cellular constituents and functions in the cells.

46. The database of claim 44, which is constructed by screening a library for molecules or agents that have an effect on the interaction between two or more molecules of interest.

47. The database of claim 44, which is constructed by screening a library for molecules or agents that disrupt the interaction between two or more cellular molecules.

48. A biosensor comprising a polypeptide comprising a sequence of amino acids comprising SEQ ID NO: 8, 10, or 12.

49. A nucleic acid encoding the biosensor of claim 48.

50. The nucleic acid of claim 49, which comprises a DNA sequence comprising SEQ ID NO: 7, 9, or 11.


SEQUENCE LISTING (12 sequences)

SEQ ID NO: 1 - protein, 19 aa, Artificial Sequence
  Synthetically created peptide
KRTADGSEFESPKKARKVE

SEQ ID NO: 2 - protein, 20 aa, Artificial Sequence
  Synthetically created peptide
QQMGRGSEFEPAAKRAKLDE

SEQ ID NO: 3 - protein, 19 aa, Artificial Sequence
  Synthetically created peptide
QQMGRGSEFESPKKARKVE

SEQ ID NO: 4 - protein, 18 aa, Artificial Sequence
  Synthetically created peptide
NSNELALKLAGLDINKTE

SEQ ID NO: 5 - protein, 18 aa, Artificial Sequence
  Synthetically created peptide
HAEKVAEKLEALSVKEET

SEQ ID NO: 6 - protein, 18 aa, Artificial Sequence
  Synthetically created peptide
PSTRIQQQLGQLTLENLQ

SEQ ID NO: 7 - DNA, 1338 bp, Artificial Sequence
  Synthetically created nucleic acid sequence encoding GFP-NES-NLS-p53
atggtgaaccggaacgtgctgaagaacaccggcctgaaggagatcatgagcgccaaggccagcgtggagggcatcgtgaacaaccacgtgttcagcatggagggcttcggcaagggcaacgtgctgttcggcaaccagctgatgcagatccgggtgaccaagggcggccctctgcccttcgccttcgacatcgtgagcatcgccttccagtacggcaaccggaccttcaccaagtatcccgacgacatcgccgactacttcgtgcagagcttccctgccggcttcttctacgagcggaacctgcggttcgaggacggcgccatcgtggacatccggagcgacatcagcctggaggacgacaagttccactacaaggtggagtaccgcggcaacggcttccctagcaacggccctgtgatgcagaaggccatcctgggcatggagcccagcttcgaggtggtgtacatgaacagcggcgtgctggtgggcgaggtggacctggtgtacaagctggagagcggcaactactacagctgccacatgaagaccttctaccggagcaagggcggcgtgaaggagttccctgagtaccacttcatccaccaccggctggagaagacctacgtggaggagggcagcttcgtggagcagcacgagaccgccatcgcccagctgaccaccatcggcaagcctctgggcagcctgcacgagtgggtggaattcacgcgtggtacctctagacctcagactccactgcacaccagccgtgtcctgaaggaggacaaggaacgatgggaggatgtcaaggaggagatgaccagtgccttggccacgatgtgtgttgactatgagcagatcaagataaagaagatagaagacgcatccccaaagaagaagcgaaaggtgctcgagatggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccgcgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgctaa

SEQ ID NO: 8 - protein, 445 aa, Artificial Sequence [sequence omitted]

SEQ ID NO: 9 - DNA, 1590 bp, Artificial Sequence
  Synthetically created nucleic acid sequence encoding HDM2-NES-myc
atgtgcaataccaacatgtctgtacctactgatggtgctgtaaccacctcacagattccagcttcggaacaagagaccctggttagaccaaagccattgcttttgaagttattaaagtctgttggtgcacaaaaagacacttatactatgaaagaggttcttttttatcttggccagtatattatgactaaacgattatatgatgagaagcaacaacatattgtatattgttcaaatgatcttctaggagatttgtttggcgtgccaagcttctctgtgaaagagcacaggaaaatatataccatgatctacaggaacttggtagtagtcaatcagcaggaatcatcggactcaggtacatctgtgagtgagaacaggtgtcaccttgaaggtgggagtgatcaaaaggaccttgtacaagagcttcaggaagagaaaccttcatcttcacatttggtttctagaccatctacctcatctagaaggagagcaattagtgagacagaagaaaattcagatgaattatctggtgaacgacaaagaaaacgccacaaatctgatagtatttccctttcctttgatgaaagcctggctctgtgtgtaataagggagatatgttgtgaaagaagcagtagcagtgaatctacagggacgccatcgaatccggatcttgatgctggtgtaagtgaacattcaggtgattggttggatcaggattcagtttcagatcagtttagtgtagaatttgaagttgaatctctcgactcagaagattatagccttagtgaagaaggacaagaactctcagatgaagatgatgaggtatatcaagttactgtgtatcaggcaggggagagtgatacagattcatttgaagaagatcctgaaatttccttagctgactattggaaatgcacttcatgcaatgaaatgaatcccccccttccatcacattgcaacagatgttgggcccttcgtgagaattggcttcctgaagataaagggaaagataaaggggaaatctctgagaaagccaaactggaaaactcaacacaagctgaagagggctttgatgttcctgattgtaaaaaaactatagtgaatgattccagagagtcatgtgttgaggaaaatgatgataaaattacacaagcttcacaatcacaagaaagtgaagactattctcagccatcaacttctagtagcattatttatagcagccaagaagatgtgaaagagtttgaaagggaagaaacccaagacaaagaagagagtgtggaatctagtttgccccttaatgccattgaaccttgtgtgatttgtcaaggtcgacctaaaaatggttgcattgtccatggcaaaacaggacatcttatggcctgctttacatgtgcaaagaagctaaagaaaaggaataagccctgcccagtatgtagacaaccaattcaaatgattgtgctaacttatttccccatgtctactgtccacgaaatcctgtgcaagctcagcttggagggtgttcattctacacccccaagtgccgaacaaaaactcatctcagaagaggatctgaatatgcataccggttag

SEQ ID NO: 10 - protein, 529 aa, Artificial Sequence
  Synthetically created polypeptide HDM2-NES-myc
MCNTNMSVPTDGAVTTSQIPASEQETLVRPKPLLLKLLKSVGAQKDTYTMKEVLFYLGQYIMTKRLYDEKQQHIVYCSNDLLGDLFGVPSFSVKEHRKIYTMIYRNLVVVNQQESSDSGTSVSENRCHLEGGSDQKDLVQELQEEKPSSSHLVSRPSTSSRRRAISETEENSDELSGERQRKRHKSDSISLSFDESLALCVIREICCERSSSSESTGTPSNPDLDAGVSEHSGDWLDQDSVSDQFSVEFEVESLDSEDYSLSEEGQELSDEDDEVYQVTVYQAGESDTDSFEEDPEISLADYWKCTSCNEMNPPLPSHCNRCWALRENWLPFDKGKDKGEISEKAKLENSTQAEEGFDVPDCKKTIVNDSRESCVEENDDKITQASQSQESEDYSQPSTSSSIIYSSQEDVKEFEREETQDKEESVESSLPLNAIEPCVICQGRPKNGCIVHGKTGHLMACFTCAKKLKKRNKPCPVCRQPIQMIVLTYFPMSTVHEILCKLSLEGVHSTPPSAEQKLISEEDLNMHTG

SEQ ID NO: 11 - DNA, 1545 bp, Artificial Sequence
  Synthetically created nucleic acid sequence encoding HDM2-NES
atgtgcaataccaacatgtctgtacctactgatggtgctgtaaccacctcacagattccagcttcggaacaagagaccctggttagaccaaagccattgcttttgaagttattaaagtctgttggtgcacaaaaagacacttatactatgaaagaggttcttttttatcttggccagtatattatgactaaacgattatatgatgagaagcaacaacatattgtatattgttcaaatgatcttctaggagatttgtttggcgtgccaagcttctctgtgaaagagcacaggaaaatatataccatgatctacaggaacttggtagtagtcaatcagcaggaatcatcggactcaggtacatctgtgagtgagaacaggtgtcaccttgaaggtgggagtgatcaaaaggaccttgtacaagagcttcaggaagagaaaccttcatcttcacatttggtttctagaccatctacctcatctagaaggagagcaattagtgagacagaagaaaattcagatgaattatctggtgaacgacaaagaaaacgccacaaatctgatagtatttccctttcctttgatgaaagcctggctctgtgtgtaataagggagatatgttgtgaaagaagcagtagcagtgaatctacagggacgccatcgaatccggatcttgatgctggtgtaagtgaacattcaggtgattggttggatcaggattcagtttcagatcagtttagtgtagaatttgaagttgaatctctcgactcagaagattatagccttagtgaagaaggacaagaactctcagatgaagatgatgaggtatatcaagttactgtgtatcaggcaggggagagtgatacagattcatttgaagaagatcctgaaatttccttagctgactattggaaatgcacttcatgcaatgaaatgaatcccccccttccatcacattgcaacagatgttgggcccttcgtgagaattggcttcctgaagataaagggaaagataaaggggaaatctctgagaaagccaaactggaaaactcaacacaagctgaagagggctttgatgttcctgattgtaaaaaaactatagtgaatgattccagagagtcatgtgttgaggaaaatgatgataaaattacacaagcttcacaatcacaagaaagtgaagactattctcagccatcaacttctagtagcattatttatagcagccaagaagatgtgaaagagtttgaaagggaagaaacccaagacaaagaagagagtgtggaactcagtttgccccttaatgccattgaaccttgtgtgatttgtcaaggtcgacctaaaaatggttgcattgtccatggcaaaacaggacatcttatggcctgctttacatgtgcaaagaagctaaagaaaaggaataagccctgcccagtatgtagacaaccaattcaaatgattgtgctaacttatttccccatgtctactgtccacgaaatcctgtgcaagctcagcttggagggtgttcattctacacccccaagtgcctag

SEQ ID NO: 12 - protein, 514 aa, Artificial Sequence
  Synthetically created polypeptide HDM2-NES
MCNTNMSVPTDGAVTTSQIPASEQETLVRPKPLLLKLLKSVGAQKDTYTMKEVLFYLGQYIMTKRLYDEKQQHIVYCSNDLLGDLFGVPSFSVKEHRKIYTMIYRNLVVVNQQESSDSGTSVSENRCHLEGGSDQKDLVQELQEEKPSSSHLVSRPSTSSRRRAISETEENSDELSGERQRKRHKSDSISLSFDESLALCVIREICCERSSSSESTGTPSNPDLDAGVSEHSGDWLDQDSVSDQFSVEFEVESLDSEDYSLSEEGQELSDEDDEVYQVTVYQAGESDTDSFEEDPEISLADYWKCTSCNEMNPPLPSHCNRCWALRENWLPEDKGKDKGEISEKAKLENSTQAEEGFDVPDCKKTIVNDSRESCVEENDDKITQASQSQESEDYSQPSTSSSIIYSSQEDVKEFEREETQDKEESVESSLPLNAIEPCVICQGRPKNGCIVHGKTGHLMACFTCAKKLKKRNKPCPVCRQPIQMIVLTYFPMSTVHEILCKLSLEGVHSTPPSA